(19) United States
(12) Patent Application Publication
(10) Pub. No.: US 2022/0280942 A1
(43) Pub. Date: Sep. 8, 2022

(54) ENZYMATIC SAMPLE PURIFICATION

(71) Applicant: Hewlett-Packard Development Company, L.P., Spring, TX (US)

(72) Inventors: Viktor Shkolnikov, Palo Alto, CA (US); Anita ROGACS, San Diego, CA (US)

(73) Assignee: Hewlett-Packard Development Company, L.P., Spring, TX (US)

(21) Appl. No.: 17/752,394

(22) Filed: May 24, 2022

Related U.S. Application Data
(63) Continuation-in-part of application No. 15/770,479, filed on Apr. 23, 2018.

(51) Int. Cl. B01L 3/00; G01N 1/34; C07K 1/34; B01L 7/00

(52) U.S. Cl. CPC B01L 3/502753; G01N 1/34; C07K 1/34; B01L 3/502715; B01L 7/52; G01N 2001/4038

(57) ABSTRACT

An apparatus of an enzymatic sample purifier can include a reaction chamber containing a polypeptide synthesizing enzyme capable of reacting with a protein degradation product to form polypeptides having a size of at least 1,000 Da and including the protein degradation product, the reaction chamber having a port configured to receive a fluid sample including a target analyte having a size of no greater than 500 Da and the protein degradation product. The apparatus can also include a product chamber, an electrophoretic transport mechanism to move a purified portion of the fluid sample including the target analyte to the product chamber, and a separator positioned to retain the formed polypeptides resulting from reaction of the protein degradation product with the polypeptide synthesizing enzyme in the reaction chamber and to pass the target analyte to the product chamber, wherein the separator comprises a porous matrix.

This application is a continuation-in-part of U.S. patent application Ser. No. 15/770,479, filed on Apr. 23, 2018.

### BACKGROUND

Fluid samples, such as blood, serum, cell lysate, extracellular fluid, milk, soils dispersed in a carrier, and the like can be analyzed to identify target analytes or characterize target analytes within the fluid sample. Identifying or characterizing the analytes amongst multiple molecules of fluid samples is often difficult and time-consuming as other constituents therein can interfere with the identification or characterization of a target analyte.

### BRIEF DESCRIPTION OF THE DRAWINGS

FIG. 1 is a schematic diagram of an example enzymatic sample purifier;

FIG. 2 is a flow diagram of an example enzymatic sample purification method;

FIG. 3 is a schematic diagram illustrating an example enzymatic sample purification method;

FIG. 4 is a schematic diagram of an example enzymatic sample purifier;

FIG. 5 is a schematic diagram illustrating an example enzymatic sample purification method; and

FIG. 6 is a schematic diagram of an example enzymatic sample purifier.

### DETAILED DESCRIPTION

A fluid sample, such as blood, serum, cell lysate, extracellular fluid, milk, soils suspended in fluid, and/or the like can contain degradation products of protein, such as amino acids, peptides and/or polypeptides, which can interfere with the detection and sensing of a target analyte in a fluid sample. Because these protein degradation products may have sizes and other properties similar to that of a target analyte, separation and removal of such protein degradation products may be difficult, costly, and time consuming.

Examples of enzymatic sample purification apparatuses, systems, and methods that utilize enzymes and can facilitate faster and less costly preparation of a fluid sample for analysis are disclosed herein. Thus, enzymatic sample purifiers and/or apparatuses (or subcomponents) of enzymatic sample purifiers and can utilize enzymes that can increase a reaction rate with carboxylic acids and amines to form peptide bonds. The enzymes can permit protein degradation products which include carboxylic acid functional groups, amine functional groups, or combinations thereof to create a formed polypeptide having a size larger than a size of the target analyte in the fluid sample.

In some examples, an apparatus of an enzymatic sample purifier includes a reaction chamber containing a polypeptide synthesizing enzyme capable of reacting with a protein degradation product to form polypeptides having a size of at least 1,000 Da that includes the protein degradation product. For example, the reaction chamber includes a port configured to receive a fluid sample including a target analyte having a size of no greater than 500 Da as well as the protein degradation product, a product chamber, and electrophoretic transport mechanism to move a purified portion of the fluid sample including the target analyte to the product chamber. The reaction chamber in this example also includes a separator positioned to retain the formed polypeptides resulting from reaction of the protein degradation product with the polypeptide synthesizing enzyme in the reaction chamber and to pass the target analyte to the product chamber, wherein the separator comprises a porous matrix. In some examples, the separator can be selected from a group of separators, such as a size exclusion chromatography separator, a dialysis separator, an electro-dialysis separator, and/or an electrophoresis separator. In other examples, the apparatus can include a receiver interconnected to the reaction chamber and the product chamber. The receiver can be sized to removably receive and retain the separator between the reaction chamber and the product chamber. The apparatus ca also include a first capture moiety in the reaction chamber to be incorporated into the formed polypeptides, and a second capture moiety carried by the separator, the second capture moiety to bind with the first capture moiety of the formed polypeptides. The second capture moiety can be fixed to the porous matrix, for example. The first capture moiety can include the amino acid histidine and the second capture moiety can include nickel. In other examples, the first capture moiety can include a first chemical composition, the second capture moiety can include a second chemical composition different than the first chemical composition, and the second chemical composition can be configured to directly bind to the first capture moiety.

In other examples, an enzymatic sample purifier can include a reaction chamber, a porous matrix, a product chamber, and a transport mechanism, and a controller. The reaction chamber in this example contains a polypeptide synthesizing enzyme and includes a port configured to receive a fluid sample comprising a protein degradation product of a peptide and a target analyte. The porous matrix retains formed polypeptides resulting from a reaction of the peptide of the protein degradation product with the polypeptide synthesizing enzyme in the reaction chamber while allowing purified portions of the fluid sample to pass therethrough, The product chamber receives the purified portions of the fluid sample including the target analyte from the reaction chamber that have passed through the porous matrix, and the transport mechanism moves the purified portions of the fluid sample including the target analyte from the reaction chamber to the product chamber. The transport mechanism also includes an electrophoretic transport mechanism, a pressurized liquid flow transport mechanism, or the combination thereof. The controller is included in this example to activate the transport mechanism. In some examples, the controller can be operable to control a temperature of the reaction chamber and in addition to activating the transport mechanism. Thus, there may be a temperature control system as well that includes a heater to heat the contents of the reaction chamber, and a sensor to sense a temperature of the contents of the reaction chamber. The controller can thus be further configured to control actuation of the heater based upon signals from the sensor.

In other examples, an apparatus of an enzymatic sample purifier includes a reaction chamber containing a polypeptide synthesizing enzyme having characteristics to react with a protein degradation product to formed polypeptides of the protein degradation product. The reaction chamber has a port configured to receive a fluid sample comprising the protein degradation product and a target analyte. The apparatus in this example also includes a product chamber and a separator to retain the larger sized polypeptides resulting from reaction of the protein degradation product with the polypeptide synthesizing enzyme in the reaction chamber and to pass purified remaining portions of the fluid sample comprising the target analyte to the product chamber. The apparatus also includes a first capture moiety in the reaction chamber to be incorporated into formed polypeptides as well as a second capture moiety carried by the separator to bind with the first capture moiety of the formed polypeptides. The separator can include a porous matrix, for example. The first capture moiety can included the amino acid histidine and the second capture moiety can include nickel in some examples. In some examples, the apparatus can include a transport mechanism, which may be controlled by a controller as part of an enzymatic sample purifier or enzymatic sample purification system.

In other examples, a method of purifying a sample can include depositing a sample comprising a target analyte and a protein degradation product into a reaction chamber containing an enzyme to form a solution in the reaction chamber, reacting the protein degradation product with the enzyme to produce a formed polypeptide, and separating the formed polypeptide and the target analyte separating the formed polypeptide and the target analyte based on differences between the formed polypeptide and the target analyte. In some examples, separating the formed polypeptide and the target analyte can include retaining the formed polypeptide within the reaction chamber and transporting the target analyte to a separate product chamber. For example, retaining the formed polypeptide can be by a porous matrix separating the reaction chamber from the product chamber, or applying an electric field to the solution such that the electric field moves the polypeptide and the target analyte at different rates in response to the size differences between the polypeptide and the target analyte. In other examples, the method can include sensing a temperature of the reaction chamber, and activating a heater to apply heat to the reaction chamber based upon the sensed temperature of the reaction chamber.

FIG. 1 schematically illustrates an example enzymatic sample purifier 20 which may facilitate faster, less costly, and more effective isolation of a target analyte that does not have accessible carboxylic acid functional groups, amine functional groups, or both accessible carboxylic acid functional groups and amine functional groups for subsequent analysis. The example enzymatic sample purifiers (or purification systems and methods herein), for example, can utilize differences between the target analyte and the formed polypeptide to separate the formed polypeptide from the target analyte allowing for subsequent analysis of the target analyte without interference from the protein degradation products. As described herein, an enzymatic sample purifier includes a reaction chamber, a product chamber, and a controller. An apparatus that can be used as part of an enzymatic sample purifier can include the reaction chamber and the product chamber, and typically includes another component, such as a separator, transport mechanism, and/or the like, which can be controlled by the controller of the enzymatic sample purifier.

In this example, enzymatic sample purifier 20 purifies the fluid sample by allowing enzymes that can react with protein degradation products such as small peptide chains, carboxylic acids, and/or amine groups to react and create formed polypeptides. Following the reaction, the enzymatic sample purifier 20 separates the formed polypeptides from the target analyte. As shown by FIG. 1, enzymatic sample purifier 20 comprises a reaction chamber 24, a product chamber 26, and a separator (S) 30. The separator can be positioned between the reaction chamber and the product chamber and can be sized or treated to retain the formed polypeptides in the reaction chamber or the separator while permitting the target analyte to pass through the separator into the product chamber.

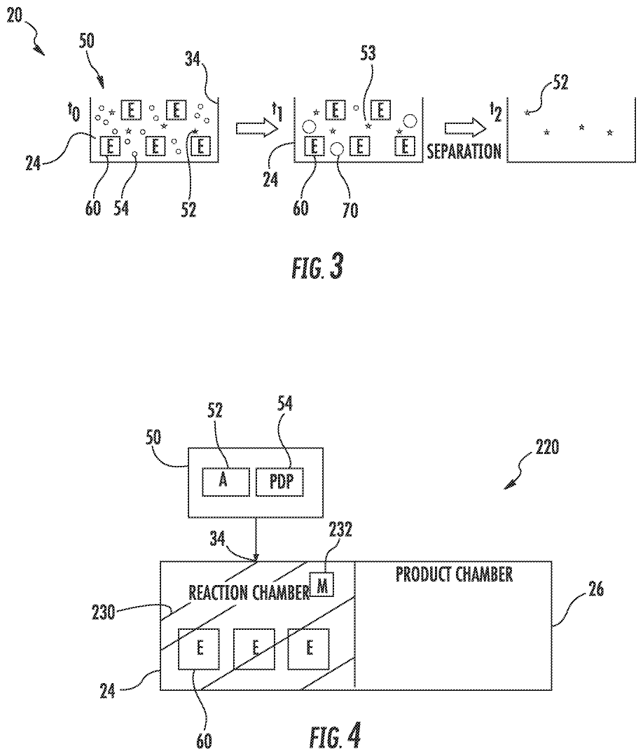

Reaction chamber 24, in further detail, comprises a chamber with a port 34. The port can be sized and shaped to allow for insertion of a fluid sample into the chamber. The chamber can be sized and shaped to receive and contain a fluid sample 50 including a target analyte (A) 52 and a protein degradation product (PDP) 54, such as carboxylic acids, amine groups, and/or peptides (schematically shown in FIGS. 3 and 5). The reaction chamber 24 further contains a polypeptide synthesizing enzyme (“enzyme”) 60 therein. The term “polypeptide synthesizing enzyme” is inclusive of “protein synthesizing enzyme,” as a polypeptide is typically understood to refer to a chain of many amino acids, whereas a protein is typically understood to contain one or more polypeptides. Therefore, both polypeptides and proteins are long chains of amino acids held together by peptide bonds. In accordance with this, enzyme 60 can be chosen based upon the characteristics of the target analyte 52 in the fluid sample 50 to be analyzed. Enzyme 60 is selected to react with a PDP in the fluid sample and create formed polypeptides from the PDP, while not reacting with target analyte 52.

The enzymes are catalytic enzymes. These enzymes increase the rate of a process by molecules. The enzymes allow for initiation of reactions and elongation of the protein degradation products to create formed polypeptides. Examples of different polypeptide synthesizing enzymes 60 include decarboxylases, polymerases, transferases, carbodiimides, dehydrogenases, isomareses, mutases, kinases, lipases, ligases, synthetases, synthases, lyases, transaminases, nucleases, and under controlled conditions, proteases. In an example, the polypeptide synthesizing enzyme can include decarboxylases, polymerases, transferases, carbodiimides, dehydrogenases, isomareses, mutases, kinases, lipases, ligases, synthetases, synthases, lyases, transaminases, and nucleases. In some examples, the polypeptide synthesizing enzymes can include synthetases. In another example, the polypeptide synthesizing enzymes can include synthases. Example syntheses include ATP synthase, citrate synthase, tryptophan synthase, pseudouridine synthase, fatty acid synthase, and cellulose synthase. Proteases can be used under controlled conditions when they would function to catalyze a synthesis process. This is opposite their normal function. When proteases are used they can include pepsin, papain, trypsin, elastase and others. The enzyme in the reaction chamber may depend on the target analyte 60 and the protein degradation products in the fluid sample. In some examples, the reaction chamber can also include carboxylic acid groups, and/or amine groups to serve as a supplementary reactant source.

As illustrated by dashed lines in FIG. 1, in some implementations, the reaction chamber 24 may contain other elements such as enzyme co-factors (CF), co-solvents (CS) and buffers (B) 61 (designated by the abbreviations CF, CS, and B in FIG, 1), Enzyme co-factors are non-protein molecules that catalyze reactions between the enzymes and the protein degradation product 54.

Examples of enzyme co-factors can include inorganic ions, organic molecules, or combinations thereof. Inorganic ions can include zinc or cupric ions. Organic molecules can include vitamins, biotin, folic acid, pantothenic acid, or combinations thereof.

Co-solvents, as used herein, can include any liquid capable of suspending the polypeptide synthesizing enzyme 60 and dissolving other substances present in a reaction chamber, such as co-factors, to form a solution. In one implementation, the co-solvents may be chosen to enhance the reactivity of polypeptide synthesizing enzyme 60 with protein degradation product 54. In one implementation, co-solvents may be organic. In other implementations, co-solvents may be inorganic. Examples of co-solvents include, but are not limited to, water, saline, and alcohols. Example alcohols can include methanol, ethanol, ethylene glycol, propylene glycol, glycerol, and combinations thereof.

Buffers include molecules that can inhibit changes to the pH of the solution. Such buffers may serve as a means of keeping pH at a nearly constant value. Examples of buffers include, but are not limited to, carbonic acid, bicarbonate, phosphate, sodium hydroxide, sodium acetate, and the like. Each of the enzyme co-factors, co-solvents, and buffers may be chosen to facilitate thermodynamic conditions favorable for the formation of amide bonds, rather than the breakage thereof, to create the formed polypeptides. The co-factors, co-solvents, and buffers may be preloaded into the reaction chamber or can be added thereto when the fluid sample can be loaded therein.

In one implementation, the reaction chamber 24 can be prefilled with the polypeptide synthesizing enzyme 60, as well as enzyme co-factors, co-solvents and buffers, wherein reaction chamber 24 can be “factory sealed” until use. A pre-filled enzymatic sample purifier may further include preservatives to maintain the function of the protein degradation products, enzyme co-factors, co-solvents, and buffers therein. A pre-filled enzymatic sample purifier 20 may be labeled for use with a particular type of sample 50, a particular type of protein degradation product 54 and/or a particular type of target analyte 52. The reaction chamber 24 may be prefilled with the corresponding type and amount of a selected polypeptide synthesizing enzyme 60, as well as respective co-factors, co-solvents and buffers suitable for the fluid sample 50, based on a particular type of protein degradation product 54 and/or the particular type of target analyte 52 contained therein. As a result, different enzymatic sample purifiers 20 with different combinations of prefilled solution mixes within reaction chamber 24 may be specifically tuned for the exclusion of particular protein degradation product 54, particular amino acids, and/or oligopepetides. Mass preparation of the enzymatic sample purifier under controlled manufacturing specifications can reduce or eliminate the task of an individual preparing the enzymatic “cocktail” used in the enzymatic sample purifier and can potentially reduce technician error. Moreover, the time and cost associated with individually preparing an enzymatic cocktail to purify fluid sample 50 can be reduced. A self-contained enzymatic sample purifier 20 may be adapted for portable applications.

Product chamber 26 can comprise a chamber that can be sized to receive the target analyte 52 after the target analyte 52 has been separated from protein degradation product 54 in the fluid sample 50 by separator 30. Product chamber 26 can be connected to reaction chamber 24, directly or indirectly. In an example, the product chamber can be directly connected to the reaction chamber. A removable separator can be slid in between numbs, or some other type of holder positioned on a wall of a chamber to separate the reaction chamber from the product chamber. In one implementation, product chamber 26 can be indirectly connected to reaction chamber 24, wherein separator 30 can extend between and can be connected to opposite sides of reaction chamber 24 and product chamber 26. In some implementations, product chamber 26 may contain a chemical solution selected to preserve or otherwise further prepare the extracted target analyte 52 for analysis.

Separator 30 can include a structure or device that can isolate the target analyte 52 from the formed polypeptide resulting from the reaction of enzyme 60 with protein degradation product 54 in reaction chamber 24. The separator can isolate formed polypeptides based on size, by adhesion, or a combination thereof. In one implementation, separator 30 may include a structure or device that separates target analyte 52 from the formed polypeptides based on size differences between molecules of target analyte 52 and molecules of formed polypeptides. A size-based separator can be selected from a group of size-based separators consisting of a size exclusion chromatography separator, a dialysis separator, an electro-dialysis separator, and an electrophoresis separator. In other implementations, separator 30 may utilize other size-based separation technology.

For example, a sized based separator can include pores having a size larger than the target analyte and can have pores that can be smaller than a size of the formed polypeptides or can provide steric hindrance to the formed polypeptides thereby preventing passage of the formed polypeptides therethrough. Size based separators are labeled to exclude molecules having a certain size. The exclusion labeling can exclude molecules having a size ranging from greater than 500 Da to 1,000,000 Da, from 750 Da to 1,000,000 Da, from 1,000 Da to 1,000,000 Da, from 750 Da to 750,000 Da, or from 1,000 Da to 500,000 Da, for example. In another implementation, separator 30 can include factors bound thereto that can be selected to bind with and trap formed polypeptides in the separator. For example, the reaction chamber 24 may comprise a first capture moiety while separator 30 may comprise and/or fixedly support a second capture moiety. The first and second capture moieties can become bound to one another. The first capture moiety contained within reaction chamber 24 can react with protein degradation products and become incorporated with the formed polypeptides. The first capture moiety can be configured to react with the second capture moiety. As a result, the formed polypeptides, including the first capture moiety, can become combined with the second capture moiety of the separator 30, thereby inhibiting the flow of the formed polypeptides through the separator 30 into the product chamber. The first and second capture moieties can be un-reactive with the target analyte. The target analyte 52 can be permitted to pass through the separator 30 into the product chamber 26.

When the separation is based on capture moieties, the reaction chamber 24 can comprise a first capture moiety, such as an amino acid histidine, The separator 30 can comprise a porous matrix or other membrane having immobilized nickel thereon. The amino acid histidine can become incorporated into the formed polypeptides and can bind with the nickel on the separator. The binding can prevent the formed polypeptide from passing through the separator into the product chamber.

In one implementation, separator 30 can be integrally formed as a single unitary body with reaction chamber 24 and product chamber 26. The separator can be permanently fixed between reaction chamber 24 and product chamber 26. For example, the separator 30 may comprise a membrane or filter which can allow molecules of a certain size to pass therethrough. The membrane or filter can allow the smaller sized target analyte molecules 52 to pass from reaction chamber 24, across separator 30, into product chamber 26, while the formed polypeptides incorporating the protein degradation product 54 can be inhibited from passing through separator 30 into product chamber 24.

In yet another implementation, separator 30 may comprise a removable unit or component of the enzymatic sample purifier 20. The term “releasably” or “removably” with respect to an attachment or coupling of two structures means that the two structures may be repeatedly connected and/or disconnected to and from one another without material damage to either of the two structures or their functioning. For example, as indicated by broken lines in FIG. 1, in one implementation, reaction chamber 24 and product chamber 26 may be interconnected by a channel, slot or other receiver 64 that can removably receive separator 30 between reaction chamber 24 and product chamber 26. In such an implementation, receiver 64 can facilitate removal and exchange of separator 30, facilitating reuse of enzymatic sample purifier 20 when separator 30 cannot have any further use or needs to be cleaned. The modification of the enzymatic sample purifier 20 to accommodate different separators can permit the purification of different fluid samples 50.

In one implementation, receiver 64 can facilitate the swapping of different separators 30. The different separators 30 may have different filtering characteristics. The different filtering characteristics can include filters having different pore sizes, filters treated with different capture moieties, or a combination thereof. For example, different types of target analytes having different sizes may be purified from the respective fluid samples utilizing multiple separators 30 which can have different pore sizes. In another example, different types of target analytes can be separated due to differences in the protein degradation products in the sample fluid and the formed polypeptides resulting from the reaction of enzyme 60 and protein degradation product 54. For example, receiver 64 can facilitate the swapping of different separators 30, having different capture moieties that can cooperate with the corresponding capture moieties in reaction chamber 24 that can become incorporated into the formed polypeptides.

In yet other implementations, separator 30 may comprise a separate component or device, distinct from one or both of reaction chamber 24 and product chamber 26. For example, in other implementations, separator 30 may be provided as part of a single unit with reaction chamber 24, wherein the target analyte 52 can be extracted through separator 30 into a separate product chamber 26.

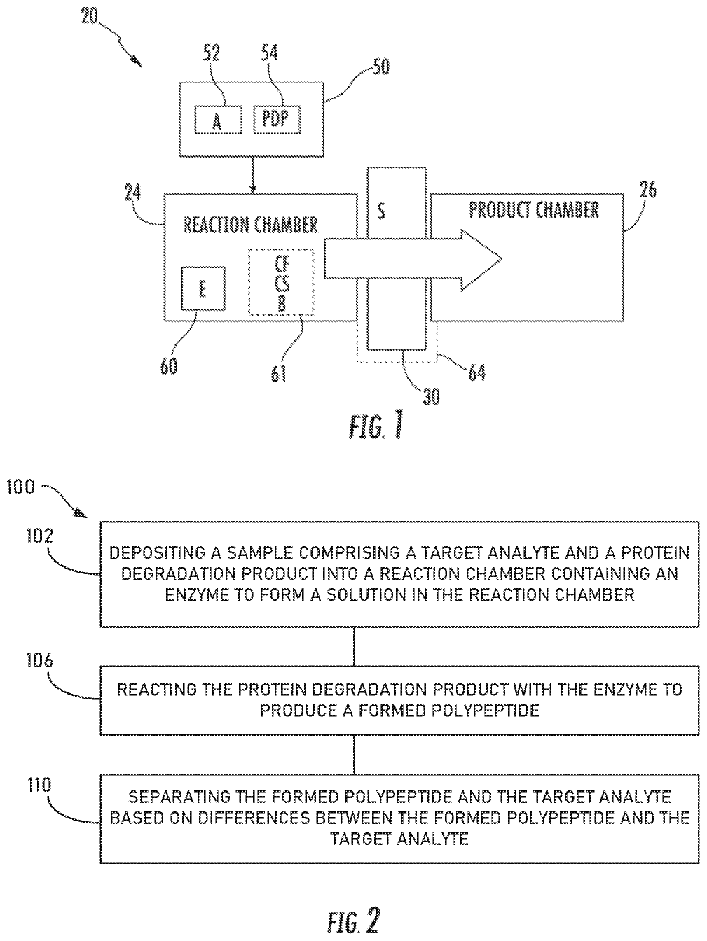

FIG. 2 and FIG. 3 illustrate an example method 100 for enzymatically purifying a fluid sample for analysis or characterization of a target analyte therein. The method may be carried out using the enzymatic sample purifier presented herein. As indicated by block 102 and illustrated at time to in FIG. 3, fluid sample 50 can be deposited through port 34 into reaction chamber 24. Fluid sample 50 can comprise target analyte 52 (schematically illustrated with stars) and protein degradation product 54 (schematically illustrated as an amino acid with solid circles), Examples of fluid sample 50 include, but are not limited to, blood, serum, cell lysate, extracellular fluid, milk, soil suspended in a carrier, and the like. In some examples, target analyte 52 may comprise small molecules that may be unable to form peptide bonds (amide bonds). For example, the target analyte may not have primary amine groups or carboxylic acid groups and can therefore be unable to form amide bonds. In other examples, the target analyte may include amine groups or carboxylic acid groups but these groups can be sterically hindered and therefore unavailable for a reaction. In one example, the target analyte can include a chlorinated polycyclic aromatic hydrocarbon, polychlorinated biphenyls, or the like.

Target analyte 52 may additionally or alternatively comprise molecules that are able to form peptide bonds, but do not form peptide bonds under the enzymatic conditions present in reaction chamber 24 due to the polypeptide synthesizing enzyme 60 chosen or the other conditions of reaction chamber 24. Such molecules may have primary amine groups or carboxylic acid groups, but may be unable to undergo peptide bond formation given the polypeptide synthesizing enzyme 60 present within the reaction chamber 24. Such an analyte may include amino acid or an oligopeptide sterically excluded from the active site of the polypeptide synthesizing enzyme. Examples of such target analytes include, but are not limited to, biomarkers such as thyrotropin-releasing hormone (a tripeptide), Leu-enkephalin, Met-enkephalin (pentapeptides), and others.

In one method, the target analyte 52 and protein degradation product 54 of fluid sample 50, prior to purification may have molecular weights and radii of gyration that overlap or that are similar to make separation by size alone difficult. In addition, in some implementations, the molecules of target analyte 52 and the molecules of protein degradation product 54 may also have similar charge, electrophoretic mobility, partition coefficient (related to hydrophobicity), protein kinase A (pKa) properties, rendering ion selective, electrophoretic and/or liquid and solid-based separation difficult.

As further indicated by block 102, reaction chamber 24, into which fluid sample 50 can be deposited, contains polypeptide synthesizing enzymes (E) 60, an enzyme co-factor and a co-solvent. In one implementation, reaction chamber 24 may further include a buffer. Polypeptide synthesizing enzyme 60, the enzyme cofactor, the cosolvent, and the buffer may be as described above.

As indicated by block 106 and illustrated at time t2, in FIG. 3, the enzyme 60 and the protein degradation product 54 can be allowed to react with one another and create formed polypeptides 70 (schematically illustrated with open circles). In one implementation, this reaction can be allowed to go to completion, reaching an equilibrium state prior to separation in block 110. In other implementations, separation in block 110 can be carried out prior to the reaction reaching completion, wherein the ongoing reactions within reaction chamber 24 can be terminated for separation.

As indicated by block 110 and illustrated at time t3 in FIG. 3, the target analyte 52 can be separated from the formed polypeptides by separator 30 based upon the differences between the formed polypeptides 70 and the target analyte 52. In one implementation, the target analyte 52 can be separated from the formed polypeptides based upon size differences between the molecules of target analyte 52 and the formed polypeptides. In one implementation, molecules of the formed polypeptides 70 can have a size greater than the size of the molecules of target analyte 52. In one implementation, the formed polypeptides 70 can have a size that can be 100 percent larger than the target analyte 52. In one implementation, formed polypeptides 70 can have a size greater than 500 Da, but more typically greater than at least 750 Da or at least 1,000 Da. In other examples, formed polypeptides can have a size ranging from 1,000 Da to 1,000,000 Da, from 1,000 Da to 500,000 Da, from 250,000 Da to 750,000 Da, from 500,000 Da to 1,000,000 Da, from 1,000 Da to 250,000 Da, or from 300,000 Da to 600,000 Da. The target analyte can have a size no greater than 500 Da. In some examples a size of the target analyte can range from 1 Da to 200 Da, from 1 Da to 100 Da, from 50 Da to 150 Da, from 100 Da to 200 Da, from about 75 Da to 150 Da, from 200 Da to 400 Da, from 250 Da to 500 Da, from 300 Da to 500 Da, or from 150 Da to 300 Da, from.

In an example, as illustrated, the target analyte 52 can be transported to product chamber 26. As a result, the target analyte 52 may be better detected and analyzed with less interference from any remaining protein degradation product 54. As described above, in some implementations, separator 30 may comprise a selectively permeable membrane or filter, wherein the target analyte 52 can be allowed to pass through the membrane or filter into product chamber 26 while the formed polypeptides 70 (and any remaining enzymes 60) can remain captured within reaction chamber 24 or adhered to the separator. In other implementations other separation techniques may be employed.

FIG. 4 schematically illustrates an alternative example enzymatic sample purifier 220, an example implementation of purifier shown at 20 in FIG. 1.

Enzymatic sample purifier 220 may facilitate faster, less costly and more effective purification of a fluid sample for analysis. Enzymatic sample purifier 220 may also purify the fluid sample by creating formed polypeptides through a reaction of a polypeptide synthesizing enzyme and protein degradation products, thereby allowing the target analyte to pass through the separator into the product chamber while retaining the formed polypeptide in the reaction chamber or trapping the formed polypeptide on or in the separator. In an example, enzymatic sample purifier 220 separates out the formed polypeptides from the target analyte in order to allow the target analyte to pass to the product chamber.

Enzymatic sample purifier 220 can be similar to enzymatic sample purifier 20 shown in FIG. 1, except that the separator can be a separator matrix 230 which can extend into the reaction chamber, rather than a separator which can merely be positioned between the reaction chamber and the product chamber. A separator matrix may extend partially into or throughout the entire reaction chamber. Porous separator matrix 230 can include a grid or matrix of a material or multiple materials disposed within and contained within reaction chamber 24. Porous separator matrix 30 comprises a matrix of interconnected internal spaces or volumes, referred to as cells or pores. In one implementation, the open celled pores of matrix 30 are sized larger than the size of the molecules of target analyte 52, but smaller than the size of the formed polypeptides, resulting from the reaction of polypeptide synthesizing enzyme 60 and protein degradation product 54. In the example illustrated, porous matrix 230 can entropically trap polypeptide synthesizing enzymes 60 within reaction chamber 24.

In one implementation, porous separator matrix 230 can be formed from or can fixedly carry a first capture moiety selected to facilitate binding with a second capture moiety that can incorporate into or can become a part of the formed polypeptides, In such an implementation, the formed polypeptides can become captured or immobilized onto separator matrix 230 as a result of the moieties incorporated into the formed polypeptides. For example, in one implementation, moiety 232 may comprise amino acid histidine while separator matrix 230 can be formed from or carries an immobilized nickel such that the formed polypeptides bind to a nickel on the separator matrix 230. In other implementations, other combinations of capture moieties, in the solution within reaction chamber 24 and upon matrix 230, may be employed. In some implementations, the separation of the formed polypeptides and the target analyte 52 can be facilitated using both size-based separation as described above and the binding between the capture moieties in the polypeptides and the capture moieties in the porous separator matrix 230.

FIG. 5 is a diagram schematically illustrating the purification of a sample 50 by an enzymatic sample purifier 220, or enzymatic sample purification system. As illustrated at time to in FIG. 5, fluid sample 50 can be deposited through port 34 into reaction chamber 24. Fluid sample 50 can comprise target analyte 52 (schematically illustrated with stars) and protein degradation product 54 (schematically illustrated with solid circles). Examples of fluid sample 50 can include, but are not limited to, blood, serum, cell lysate, extracellular fluid, milk, soil suspended in a carrier, or the like. Target analyte 52 may comprise small molecules that can be unable to form peptide bonds (amide bonds). Such molecules may not have primary amine groups or carboxylic acid groups and can therefore, be unable to form amide bonds. Alternatively, such molecules can include amine groups, carboxylic acid groups, or the combination thereof but these groups can be sterically hindered.

Target analyte 52 may additionally or alternatively comprise molecules that can be able to form peptide bonds, but do not form peptide bonds under the conditions present in reaction chamber 24 due to the polypeptide synthesizing enzyme 60 chosen or the other conditions present in the reaction chamber 24. Such molecules may have primary amine groups or carboxylic acid groups, but may be unable to undergo peptide bond formation given the polypeptide synthesizing enzyme 60 within reaction chamber 24. Such an analyte may include amino acids or oligopeptides that can be sterically excluded from the active site of the polypeptide synthesizing enzyme. Examples of such analytes can include, but are not limited to, biomarkers such as thyrotropin-releasing hormone (a tripeptide), Leu-enkephalin, Met-enkephalin (pentapeptides), and others. In one method, the target analyte 52 and protein degradation product 54 of fluid sample 50, prior to purification, have molecular weights and radii of gyration that overlap one another or that are similar to one another to make separation by size alone difficult, In addition, in some implementations, the molecules of target analyte 52 and the molecules of protein degradation product 54 may also have similar charge, electrophoretic mobility, partition coefficient (related to hydrophobicity), protein kinase A (pa) properties, rendering ion selective, electrophoretic and/or liquid and solid-based separation difficult.

As further illustrated by FIG. 5, reaction chamber 24, into which fluid sample 50 can be deposited, can contain polypeptide synthesizing enzymes (E) 60, an enzyme co-factor, and a co-solvent. In one implementation, reaction chamber 24 can additionally contain a buffer. Polypeptide synthesizing enzyme 60, the enzyme co-factor, the co-solvent, and the buffer can be as described above. The polypeptide synthesizing enzyme 60 may be captured or retained within porous separator matrix 230

As illustrated at time t2, in FIG. 5, the polypeptide synthesizing enzyme 60 and the protein degradation product 54 can be allowed to react with one another and can create formed polypeptides 70 (schematically illustrated with open circles). In one implementation, this reaction can go to completion, reaching an equilibrium state prior to separation. In other implementations, separation can be carried out prior to the reaction reaching completion, wherein the ongoing reactions within reaction chamber 24 can be terminated for separation.

As illustrated at time t3 in FIG. 5, the target analyte 52 can be separated from the formed polypeptides by porous separator matrix 230 based upon the differences between formed polypeptides 70 and target analyte 52. In one implementation, the target analyte 52 can be separated from the formed polypeptides based upon size differences between the molecules of target analyte 52 and the formed polypeptides. In one implementation, the formed polypeptides 70 can each have a size much greater than the size of the molecules of target analyte 52. In one implementation, the formed polypeptides 70 are at least 100 percent larger than target analyte 52. In one implementation, formed polypeptides 70 can have a size of at least 1,000 Da and the target analyte can have a size of no greater than 500 Da.

The target analyte 52 can be transported to product chamber 26 while the formed polypeptide can be retained in the reaction chamber. As a result, the target analyte 52 may be detected and analyzed, with less or no interference from any remaining protein degradation product 54. In one implementation, reaction chamber 24 including the formed polypeptides and target analyte 52 can be centrifuged to allow for maximum contact opportunities between the formed polypeptides and the separator matrix. The formed polypeptides can remain captured within porous matrix 230 while the target analyte 52 can be discharged from reaction chamber 230.

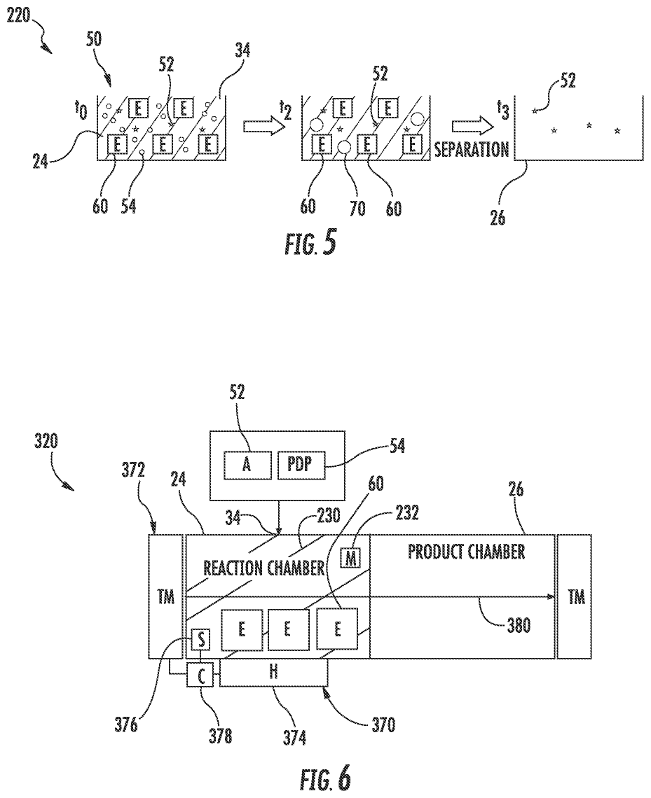

FIG. 6 schematically illustrates example enzymatic sample purification, which can be part of an enzymatic sample purifier 320, or enzymatic sample purification system. The enzymatic sample purifier 320 can additionally comprise temperature control system 370 and transport mechanism 372. Temperature control system 370 can provide closed-loop feedback for the control of the temperature of fluids within reaction chamber 24 to facilitate enzymatic reactions between polypeptide synthesizing enzyme 60 and the protein degradation product 54 of the fluid sample 50 within reaction chamber 24. Temperature control system 370 comprises heater 374, sensor 376, and controller 378. Heater 374 can include a device that can generate and emit heat so as to heat contents within reaction chamber 24. In one implementation, heater 374 can include an electrically conductive material which can produce heat through resistance to electrical current. In another implementation, heater 374 may comprise other heat generating and emitting devices. The heater can be located within the reaction chamber or on an exterior wall of the reaction chamber. In some examples, the heater can be removably coupled with the reaction chamber.

Sensor 376 can comprise a device that can sense a temperature of the fluid within the reaction chamber 24. Controller 378 can comprise a processing unit that can control the operation of heater 374 based upon signals received from sensor 376 and the sensed temperature of the content of reaction chamber 24. By controlling the temperature of the fluid within reaction chamber 24, formation of formed polypeptides through the reaction of enzymes 60 with protein degradation product 54 may be enhanced.

For purposes of this application, the term “processing unit” shall mean electronics or hardware that executes sequences of instructions contained in a non-transitory memory. Execution of the sequences of instructions causes the processing unit to perform steps such as generating control signals. The instructions may be loaded in a random access memory (RAM) for execution by the processing unit from a read only memory (ROM), a mass storage device, or some other persistent storage. In other embodiments, hard wired circuitry may be used in place of or in combination with software instructions to implement the functions described. For example, controller 378 may be embodied as part of one or more application-specific integrated circuits (ASICs). Unless otherwise specifically noted, the controller cannot be limited to any specific combination of hardware circuitry and software, nor to any particular source for the instructions executed by the processing unit.

Transport mechanism 372 can comprise a device that can move a portion of the fluid sample 50 including the target analyte from reaction chamber 24 to product chamber 26. In one implementation, transport mechanism 372 can include a pressurized liquid flow transport, wherein pressure can be applied to reaction chamber 24 to move fluid within reaction chamber 24 towards the separator and into product chamber 26 in the direction indicated by arrow 380. In another implementation, transport mechanism 372 can include an electrophoretic transport which can apply an electric field in the direction indicated by arrow 382 for movement of the fluid including the fluid sample 50, and target analyte 52, towards product chamber 26. In both implementations, the target analyte 52 can be moved towards and into product chamber 26 while the formed polypeptides from the reaction of polypeptide synthesizing enzymes 60 and protein degradation product 54 remain captured within reaction chamber 24 or at the porous separator matrix 230.

In one implementation, controller 378 can further control operation of transport mechanism 372. In one implementation, controller 378 controls the time at which transport mechanism 372 can be activated. By controlling the time at which transport mechanism 372 can be activated or the rate at which transport mechanism 372 operates, controller 378 may control the size of the formed polypeptides at the time that the fluid sample 50 can be driven towards product chamber 26. By controlling the time of the reaction, as well as the temperature in the reaction chamber, other properties of the solution in which the reactions occur, the length and size of the formed polypeptides may be controlled. As a result, controller 378 may control the temperature of reaction chamber 24 and the activation of transport mechanism 372 at a time when the formed polypeptides have a sufficient length and are sufficiently sized such that the formed polypeptides have a size or other properties to ensure they remain captured within the porous separator matrix 230 when the liquid flow bias is applied by transport mechanism 372.

As used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though the individual members of the list are individually identified as a separate and unique members. Thus, no individual member of such list should be construed as a de facto equivalent of any other member of the same list solely based on theft presentation in a common group without indications to the contrary.

Concentrations, dimensions, amounts, and other numerical data may be presented herein in a range format. A range format is used merely for convenience and brevity and should be interpreted flexibly to include the numerical values explicitly recited as the limits of the range, and also to include all the individual numerical values or sub-ranges encompassed within that range as if each numerical value and sub-range is explicitly recited. For example, a numeric range that ranges from about 10 to about 500 should be interpreted to include the explicitly recited sub-range of 10 to 500 as well as sub-ranges thereof such as about 50 and 300, as well as sub-ranges such as from 100 to 400, from 150 to 450, from 25 to 250, etc.

Although the present disclosure has been described with reference to example implementations, workers skilled in the art will recognize that changes may be made in form and detail without departing from the spirit and scope of the claimed subject matter. For example, although different example implementations may have been described as including one or more features providing one or more benefits, it is contemplated that the described features may be interchanged with one another or alternatively be combined with one another in the described example implementations or in other alternative implementations.

The following illustrate an example of the present disclosure. However, the following are illustrative of the application of the principles of the present disclosure, Numerous modifications and alternative compositions, apparatuses, methods, and/or systems may be devised without departing from the spirit and scope of the present disclosure. The appended claims are intended to cover such modifications and arrangements.

### EXAMPLE

Isolation of polychlorinated biphenyl compounds

A soil sample including polychlorinated biphenyls is suspended in water and loaded into the reaction chamber of an enzymatic sample purifier or system for enzymatic purification. Protein degradation products in the suspended soil sample react with synthase enzymes in the reaction chamber to create formed polypeptides. A pressure gradient is applied and the polychlorinated biphenyls pass through the separator into the product chamber. The isolated polychlorinated biphenyls can then be further analyzed.

What is claimed is:

1. An apparatus of an enzymatic sample purifier, comprising:
a reaction chamber containing a polypeptide synthesizing enzyme capable of reacting with a protein degradation product to form polypeptides having a size of at least 1,000 Da and including the protein degradation product, the reaction chamber having a port configured to receive a fluid sample including a target analyte having a size of no greater than 500 Da and the protein degradation product;
a product chamber;
an electrophoretic transport mechanism to move a purified portion of the fluid sample including the target analyte from the reaction chamber to the product chamber; and
a separator positioned to retain the formed polypeptides resulting from reaction of the protein degradation product with the polypeptide synthesizing enzyme in the reaction chamber and to pass the target analyte to the product chamber, wherein the separator comprises a porous matrix.

2. The apparatus of claim 1, wherein the separator is selected from a group of separators including a size exclusion chromatography separator, a dialysis separator, an electro-dialysis separator, an electrophoresis separator, or a combination thereof.

3. The apparatus of claim 1, further comprising a receiver interconnected to the reaction chamber and the product chamber, the receiver sized to removably receive and retain the separator between the reaction chamber and the product chamber.

4. The apparatus of claim 1, further comprising:
a first capture moiety in the reaction chamber to be incorporated into the formed polypeptides; and
a second capture moiety carried by the separator, the second capture moiety to bind with the first capture moiety of the formed polypeptides.

5. The apparatus of claim 4, wherein the second capture moiety is fixed to the porous matrix.

6. The apparatus of claim 4, wherein the first capture moiety comprises amino acid histidine and wherein the second capture moiety comprises nickel.

7. The apparatus of claim 4, wherein:
the first capture moiety has a first chemical composition;
the second capture moiety has a second chemical composition different than the first chemical composition; and
the second chemical composition is configured to directly bind to the first capture moiety.

8. The apparatus of claim 7, wherein the first capture moiety comprises amino acid histidine and wherein the second capture moiety comprises nickel.

9. An enzymatic sample purifier, comprising:
a reaction chamber containing polypeptide synthesizing enzyme, the reaction chamber having a port configured to receive a fluid sample comprising a protein degradation product that includes a peptide and a target analyte;
a porous matrix to retain formed polypeptides resulting from a reaction of the peptide of the protein degradation product with the polypeptide synthesizing enzyme in the reaction chamber while allowing purified portions of the fluid sample to pass therethrough;
a product chamber to receive the purified portions of the fluid sample including the target analyte from the reaction chamber that have passed through the porous matrix;
a transport mechanism to move the purified portions of the fluid sample including the target analyte from the reaction chamber to the product chamber, wherein the transport mechanism includes an electrophoretic transport mechanism, a pressurized liquid flow transport mechanism, or the combination thereof; and
a controller to activate the transport mechanism.

10. The enzymatic sample purifier of claim 9, wherein the controller is operable to control a temperature of the reaction chamber and activate the transport mechanism.

11. The enzymatic sample purifier of claim 9, further comprising a temperature control system, wherein the temperature control system includes:
a heater to heat the contents of the reaction chamber; and
a sensor to sense a temperature of the contents of the reaction chamber,
wherein the controller is further configured to control actuation of the heater based upon signals from the sensor.

12. An apparatus of an enzymatic sample purifier, comprising:
a reaction chamber containing a polypeptide synthesizing enzyme having characteristics to react with a protein degradation product to formed polypeptides of the protein degradation product, the reaction chamber having a port configured to receive a fluid sample comprising the protein degradation product and a target analyte;
a product chamber;
a separator configured to retain the larger sized polypeptides resulting from reaction of the protein degradation product with the polypeptide synthesizing enzyme in the reaction chamber and to pass purified remaining portions of the fluid sample comprising the target analyte to the product chamber;
a first capture moiety in the reaction chamber to be incorporated into formed polypeptides; and
a second capture moiety carried by the separator, the second capture moiety to bind with the first capture moiety of the formed polypeptides.

13. The apparatus of claim 12, wherein the separator comprises a porous matrix.

14. The apparatus of claim 12, wherein the first capture moiety comprises amino acid histidine and wherein the second capture moiety comprises nickel.

15. The apparatus of claim 12, further comprising a transport mechanism including a pressured liquid flow transport mechanism, an electrophoretic transport mechanism, or a combination thereof.

16. The apparatus of claim 12, wherein:
the first capture moiety has a first chemical composition;
the second capture moiety has a second chemical composition different than the first chemical composition; and
the second chemical composition of the second capture moiety is configured to directly bind.

17. A method of purifying a sample, comprising:
depositing a sample comprising a target analyte and a protein degradation product into a reaction chamber containing an enzyme to form a solution in the reaction chamber;
reacting the protein degradation product with the enzyme to produce a formed polypeptide; and
separating the formed polypeptide and the target analyte based on differences between the formed polypeptide and the target analyte.

18. The method of claim 17, wherein separating the formed polypeptide and the target analyte comprises retaining the formed polypeptide within the reaction chamber and transporting the target analyte to a separate product chamber.

19. The method of claim 17, wherein retaining the formed polypeptide within the reaction chamber is by:
a porous matrix separating the reaction chamber from the product chamber, or
applying an electric field to the solution such that the electric field moves the polypeptide and the target analyte at different rates in response to the size differences between the polypeptide and the target analyte.

20. The method of claim 17, further comprising:
sensing a temperature of the reaction chamber; and
activating a heater to apply heat to the reaction chamber based upon the sensed temperature of the reaction chamber.
